(19) United States
(12) Patent Application Publication
(10) Pub. No.: US 2019/0083559 A1
(43) Pub. Date: Mar. 21, 2019

(54) AGENT CONTAINING FAT (OIL), WHICH CONTAINS ONION EXTRACT, THE PRODUCTION AND USE THEREOF FOR CARING, PREVENTING OR TREATING DAMAGED SKIN TISSUE, ESPECIALLY SCARRED TISSUE

(71) Applicant: MERZ PHARMA GmbH & CO. KGaA, FRANKFURT am MAIN (DE)

(72) Inventors: Valentina PASPALEEVA-KUEHN, FRANKFURT AM MAIN (DE); Rolf D. Beutler, HOECHST/HUMMETROTH (DE); Simone SCHATSCHNEIDER, WIESBADEN (DE); Martina HEBERER, HEUSENSTAMM (DE)

(21) Appl. No.: 16/138,028

(22) Filed: Sep. 21, 2018

Related U.S. Application Data
(63) Continuation of application No. 12/802,250, filed on Jun. 2, 2010, now abandoned.
Application No. 12/802,250 is a division of application No. 10/481,773, filed on Dec. 22, 2003, now abandoned.

(30) Foreign Application Priority Data
Jul. 3, 2001 (DE) 10132003.5

(51) Int. Cl. A61K 36/185; A61K 36/47; A61Q 19/00; A61K 8/97; A61Q 19/08; A61K 9/06; A61K 9/00; A61K 36/8962

(52) U.S. Cl. CPC A61K 36/185; A61K 36/47; A61Q 19/00; A61K 36/8962; A61Q 19/08; A61K 9/06; A61K 9/0014; A61K 8/97

(57) ABSTRACT

The invention relates to a novel agent containing fat (oil), which contains onion extract, in addition to the production and use thereof for caring, preventing or treating damaged akin tissue, such as after operations, biopsies, wounds, burns and other accidents, and especially scarred tissue, stretch marks, degenerative skin conditions and others. The agent is characterised in that the base thereof is based on oil, which can be used to produce, for example, cream, lotion, fluid, massage, or balm based products which contain fail (oil). Surprisingly, the onion extract, which contains water, alcohol or water-alcohol, can be integrated into a base containing oil without the effectiveness thereof being reduced or a phase separation being produced. The effectiveness thereof could be proved when the medical application thereof was monitored.

### CROSS-REFERENCE TO RELATED APPLICATIONS

This application is a divisional of and Applicants claim priority under 35 U.S.C. §§ 120 and 121 of parent U.S. patent application Ser. No. 10/481,773 filed on Dec. 22, 2003, which is the National Stage of PCT/EP02/07216 filed on Jul. 1, 2002, which claims priority under 35 U.S.C. §119 of German Application No. 101 32 003.5 filed on Jul. 3, 2001. The international application under PCT article 21(2) was not published in English.

### BACKGROUND OF THE INVENTION

### 1. Field of the Invention

The present invention relates to a novel agent containing fat (oil), which contains onion extract, and to the production and use thereof for caring for, preventing or treating damaged skin tissue, such as, for example, after operations, biopsies, cuts, burns or other accidents, and in particular scarred tissue, stretch marks, degenerative changes in skin and others. The agent is characterized in that it has an oil base and, in this respect, can be prepared, for example, on the basis of a cream, lotion, fluid, massage oil or balsam. It therefore comprises fat (oil). Surprisingly, it has been possible to incorporate onion extract, which comprises, in particular, water, alcohol, or else water-alcohol, into an oil-containing base without its effectiveness being adversely affected or phase separation arising. It was possible to demonstrate the effectiveness in medicinal application observations.

### 2. The Prior Art

Agents for treating damaged skin tissue, in particular scarred tissue, are known. In particular, gel-like products are used for this purpose. U.S. Pat. No. 5,885,581 describes such a gel-like product which comprises 20-30% by weight of polyethylene glycol 200, 0.005-0.03% by weight of preservative, 0.05-0.2% by weight of sorbic acid, 0.5-2% by weight of allantoin, 1-3% by weight of xanthan and, if desired, perfume substances and which is characterized by 5-15% by weight of a liquid onion extract (Allium cepa extract), based on an aqueous carrier in an amount of about 55-65% by weight. The product thus represents a fat (oil)-free gel and is applied externally to damaged skin tissue, in particular scarred tissue. The product is further characterized by a pH of 4.5-5.5 and a particle size of less than 50 μm.

In addition, a special plaster against scars is known which, under the name “Hansaplaat® scar reduction”, is said to lead to better healing of injured skin without active ingredient. In this connection, suitable plaster pads are placed onto the skin for about 8-10 weeks and promote the regeneration process, presumably as a result of the increased temperature level.

Under the product Kelofibrase® is known a scar cream which comprises, as active ingredient, urea and heparin sodium (60 000 I.U.) and camphor as well as customary oil/emulsifier components. This product is reportedly useful for scar treatment, for scar contractures and keloids. Here, heparin-sodium acts, as is known, as a blood-thinning agent.

Under the name Hirudoid®forte is known a gel which comprises, as active ingredient, mucopolysaccharide polysulfuric ester (445 mg, corresponding to 40 000 units in 100 g of ointment). Further ingredients are the components required for the preparation of the gel, such as isopropyl alcohol, polyacrylic acid, propylene glycol and water. Mucopolysaccharide polysulfuric esters generally have a heparinoid effect and therefore correspond to the Kelofibrase® product specified above. As well as the gel, a Hirudoid®forte ointment is also known which, as well as the abovementioned active ingredients, has a mixture of monoglycerides and diglycerides with higher fatty acids and medium-chain triglycerides etc., and isopropyl alcohol, imide urea, phenoxyethanol and water. Products of this type can be used for treatment in cases of phlebopathies, superficial phlebitides, hematomas and for loosening hard scars. The product must not be applied to damaged skin.

Under the name Hylaform® is known a gel implant which contains crosslinked hyaluronic acid which is present in an aqueous sodium-chloride-containing solution for the purpose of injection. Using such an agent, skin deformations are said to be treatable. However, acute or chronic skin disorders in the affected correction area must not be present.

Under the name Linoladiol® N is known a hydrophilic O/W cream which comprises estradiol as active ingredient in the cream base. By means of this hormone-containing preparation, as well as gynecological applications, dermatological applications, such as burns, scar treatment, atrophy of the skin, perioral dermatitis and eczema in the acute and subacute stage are also stated. In this connection, however, the application limitations known for hormone-containing products, such as side effects and/or interactions, are to be taken into consideration to a considerable degree.

The product name PC 30 V describes a liquidum which comprises horse chestnut seed dry extract and also camomile blossom dry extract in 1,3-butanediol, dexpanthenol, allantoin and odor substances. This agent is said to be useful in the treatment of skin damage, such as wound chafing of sensitive pressure points and scars by orthopedic apparatuses, and also pressure sores. An indication with regard to scars as a result of operations or other skin damage is not stated here.

The ointment Striatridin® comprises, as well as the ointment base, alkyl-branched fatty acid esters of octadecyl alcohol, amino acid sol, and ethyl nicotinate as active ingredient. This product is useful for scar treatment, for stretch marks and for loose, functionally impaired skin.

DE-A 196 28 284 describes the use of bear's garlic for the treatment of psoriasis.

DE-A 37 23 248 relates to the use of thiosulfinic acid derivatives for the treatment of inflammations. These may be obtained, inter alia, by extraction from onions and subsequent chromatography. Onion extract itself is not used here.

EP-B 429 080 relates to a preparation process for S-allylcysteine-containing products, where, for example, aqueous garlic extracts are admixed with cysteine, giving S-allylcysteine.

EP-B 364 442 relates to an oil extract from at least 3 different herbs chosen from euphorbia, veronica, yarrow, fumitory, garlic, nettle and marigold. This combination is used in the form of an oil, e.g. with paraffin, against psoriasis.

EP-B EP 201 956 relates to the extraction and chromatographic fractionation, for example, of tobacco, algae, garlic, where the specific substances obtained are reportedly used as antioxidative substances in cosmetics.

U.S. Pat. No. 6,200,570 relates to compositions comprising garlic extract and at least one further plant extract, such as aloe vera, and antiinflammatory agents, such as diclofenac with an antiallergic, analgesic effect.

JP-A 2000327535 describes a hair tonic which has, for example Allium schoenoprasum and/or other plant extracts.

JP-A 09194334 relates to a hair tonic which is effective against hair lose, which has, for example, Allium sativum and/or other plant extracts.

JP-A 08012570 and JP-A 0317413 relate to antiallergenic or antidandruff agents comprising plant extracts such as Allium sativum or Allium victorialis.

The abovementioned prior art suggests that the products which can be used for scar treatment are either in the form of a gel or, if they are not in the form of a gel, comprise active ingredients which can have considerable side effects. Thus, for example, the product containing estradiol has limited uses with regard to the hormone content. Mucopolysaccharide polysulfuric esters with their heparinoid effect can trigger hypersensitivity reactions and must under no circumstances be applied to damaged skin. The abovementioned product containing Hylaform®-hyaluronic acid is an implant which may only be suitable for the mechanical correction of skin deformations and in this respect is accompanied by no permanent effect on the skin tissue itself. Liquidum PC 30 V can only be used for the treatment of pressure point scars which are small in size, as caused, for example, by orthopedic apparatuses, and in this respect is not described for the permanent changing of damaged skin tissue by operations, injuries, burns etc.

There is therefore a need for a product which does not have the abovementioned side effects. In addition, it should not be in gel form since, as is known, fat (oil)-free gels can have a rather drying effect on the skin.

### SUMMARY OF THE INVENTION

It is therefore an object of the present invention to provide a product which has a highly compatible active ingredient and where said ingredient is incorporated into an emulsion base, as a result of which the skin will not dry out and moreover a positive, care, in particular elasticity-promoting, skincare effect can also be achieved.

This object is achieved according to the invention by an agent which has an onion extract as active ingredient and, as well as the customary additives chosen from consistency-imparting agents, dyes, antioxidants, perfume substances, humectants, preservatives, stabilizers, additional active ingredients, has a fat (oil) phase.

Preferably, a water phase and corresponding emulsifiers chosen from the group of O/W and/or W/O emulsifiers or mixtures thereof or with suitable coemulsifiers, may additionally be present.

The agents can have a total of 5-99% fat (oil) phase, 0.1-35% additives, 1-20% onion extract and the other substances, if present, in the amounts given below.

Particularly suitable agents are those which are composed of 70-99%, very particularly 80-98%, fat (oil) phase, 0.1-20%, in particular 0.1-10%, additives and 1-20%, preferably 1-10% and very particularly 2-5%, onion extract, solvents preferably being present as the remainder, such as, for example, alcohols (ethanol, isopropanol) e.g. in amounts of up to 20%.

The agent according to the invention is also preferably composed of a fat (oil) phase in an amount of 5-70%, 0.1-15% emulsifier (O/W; W/O); mixtures thereof or with coemulsifiers, 0.1-35% additives, 1-20% onion extract and as the remainder water or a water/alcohol, e.g. ethanol, isopropanol, mixtures, e.g. up to 20%.

Particularly suitable as fat (oil) phase are 5-60%, preferably 5-40% and very particularly 5-25%.

The onion extract is, in particular, an onion extract having water—or alcohol—or water-alcohol.

Using such a composition it is surprisingly possible to incorporate the onion extract into a fat (oil)-containing phase such that distribution of the extract is uniform and stable and, moreover, a drying out of the treated skin cannot take place by means of this base and thus a care, elasticity-promoting effect is also achieved, such that the treatment of the damaged areas of the skin both with regard to scarring and slackening, and also with regard to the softness of the skin can be positively influenced or prevented.

In the agent according to the invention, between 1-15%, 2-15%, in particular 5-15% of the onion extract, based on the total amount, are suitable, preferably 5-10%, in particular 8-10% and very particularly preferably 2-4% or 10%.

The additives are preferably present in an amount of 0.1-30%, in particular 0.1-25%, depending on the intended use.

The amount of emulsifiers is, in particular, 0.1-10%, in particular 1-10%, preferably 1-8% and very particularly 1-5%.

The onion extract is, in particular, an aqueous-ethanolic extract. It preferably has water and 10-15% ethanol.

It may also be advantageous to use an alcoholic onion extract. As well as alcohol, this can preferably also have 10-80%, in particular 20-60%, of a solvent chosen from triglycerides, hydrocarbons and fatty acid esters. Unless stated otherwise, the quantitative data refer to % by weight.

The fat (oil) phase is preferably chosen from hydrocarbons, fatty alcohols, ethers and esters, (poly)ol fatty acid esters, triglycerides, natural oils, natural fats, waxes, silicone oils, silicone waxes or mixtures thereof. Particular preference is given here to liquid paraffins, lactic esters, fatty alcohol ethers, evening primrose oil, silicone oil or mixtures thereof.

The W/O emulsifier advantageously has a HLB value of 1-9, in particular 1-8, preferably 2-7 and very particularly preferably 3-6, and the O/W emulsifier has a HLB value of 9-18, preferably 9-15 and in particular 9-13, or is an ionic O/W emulsifier.

As W/O emulsifiers, particular preference is given to sorbitan derivatives, polyethoxylated fatty acids/alcohols/esters/triglycerides, (poly)glyceryl derivatives, polyol esters, glucose derivatives, pentaerythritol derivatives, alkylphenols, (block) polymers, fatty acid salts, siloxanes or mixtures thereof and, of these, very particular preference is given to Abil® EM 90, Arlacel® 582 and magnesium stearate or mixtures thereof.

Here too, coemulsifiers may be present, such as, for example, Arlatone®T(V).

Particularly suitable O/W emulsifiers are polyoxyethylated products, nonionic and ionic phosphates, ionic monovalent salts, (poly)glyceryl esters, sugar esters, sterol derivatives, castor oil derivatives, siloxanes or mixtures thereof or mixtures with coemulsifiers thereof. Particularly suitable here are Tego Care® 450, Eumulgin® B1 or mixtures thereof and/or with coemulsifiers.

When O/W emulsifiers are present, very particular preference is given to stabilizers chosen from acrylamides, acrylates and polysaccharides, in particular those as described below.

It is also preferred if the agents according to the invention further have, as additives, those chosen from vitamins, electrolytes, such as, for example, magnesium sulfate or sodium chloride, allantoin, D-panthenol, hyaluronic acid, mucopolysaccharides, dyes, perfume substances, preservatives, humectants.

In particular, it is also possible for wax products to additionally be present, and or alternatively, as additive, also lecithins, in particular Phosal®50 SA.

The agent can comprise varying amounts of water, fat phase, emulsifier, additives and active ingredient (onion extract). In this connection, depending on the intended use, a lotion, a fluid, a cream or a balsam/ointment, for example, are then obtained.

The agent can thus preferably be composed of a fat (oil) phase in an amount of 5-70% by weight, in particular 5-55%, of one or more W/O emulsifiers, mixtures thereof or with coemulsifiers in a total amount of 0.5-15%, in particular 0.5-10%, and 0.1-25%, in particular 0.1-22%, of additives of the type mentioned above, and 1-15%, in particular 1-10%, of the abovementioned onion extract and, as the remainder, water or a mixture of water with alcohols, such as, for example, ethanol, isopropanol, in amounts up to 20%.

The agent can, in particular, also be composed of 5-40%, in particular 5-30%, of fat (oil) phase, 0.1-15%, in particular 1-10% of one or more O/W emulsifiers or mixtures thereof or with coemulsifiers, 0.1-32%, in particular 0.1-20%, of additives and 1-10% onion extract and, as the remainder, water or a mixture of water with alcohols, such as, for example, ethanol, isopropanol, in amounts up to 20%.

In this way, the agent for the treatment of scars or of damaged and/or slackened skin tissue can, in particular, be prepared in the form, for example, of a massage oil or balsam, lotion, fluid or cream. Thus, the use of WO emulsifiers may give rise in particular to lotions, and a balsam, and the use of O/W or W/O emulsifiers may give rise in particular to cream-like products or fluids. Depending on the field of use, it is possible to choose between these products. Of particular suitability is a O/W cream of the type mentioned above for the treatment, for example, of acne scars. If larger areas are to be treated, a lotion is advisable, which can preferably be used for the treatment and also for prevention of stretch marks or generally slackened skin. For larger areas it is likewise possible to use a balsam which is very particularly suitable for the treatment of operation scars or burn scars, cuts. Massage oil is suitable in particular for prevention or care and treatment of relatively large areas of skin, such as, for example, in the case of stretch marks.

However, the administration form is not limited to a specific indication and can be varied.

### DETAILED DESCRIPTION OF PREFERRED EMBODIMENTS

The individual ingredients are described in more detail below:

### I. Onion Extract

A suitable onion extract is, in particular, an extract from dried onions comprising water, alcohol or water-alcohol. The extraction can be carried out here with water itself or else with alcohol, optionally with the addition of solvents as specified below, or a mixture of water and one or more alcohols as specified below. The dried onions, also known as dried onion chips, obtained from the stock plant Allium cepa Linne, can here firstly be extracted with the extractant by exhaustively percolating the drug preferably at elevated temperature, e.g. at 40-90° C. The percolate can then be evaporated at a suitable, in particular increased, temperature above 30° C. under reduced pressure to give the spissum extract (thickened extract), and subsequently be dissolved in the desired solvent to give the water, alcohol or water-alcohol mixture-fluid extract. Alternatively, the fluid extract can also be obtained without prior evaporation by direct reaction with the desired solvent.

This gives a reddish-brown to brown liquid with a characteristic odor. The ratio of dry drug to fluid extract can vary here from 0.1:1 to 10:1, preferably 0.1:1 to 5:1, in particular 0.15:1 to 4:1. Very particularly preferred ratios are 0.16:1 or 4:1.

Particular preference is given to fluid extracts of said type with water/alcohol mixture or only alcohol as solvent. In the mixture, preferably 5-60%, in particular 10-50% and very particularly 10-40% or else 10-15%, especially 13%, alcohol, preferably ethanol, is present.

The alcohol is preferably chosen from ethanol, isopropanol or propanol or dihydric alcohols, such as butylene glycol, propylene glycol or mixtures thereof. Particular preference is given to ethanol.

It may also be advantageous if the onion extract which is extracted with water or other alcoholic solvents, for example those specified above, to give the alcoholic fluid extract as described, is worked up. In particular, in this respect, 10-80%, preferably 20-60%, of the alcohol can be replaced by a solvent chosen from the substances specified below under point II, in particular hydrocarbons, triglycerides, particularly medium-chain ones, and fatty acid esters or mixtures thereof.

An onion extract which can be used according to the invention can, for example, be prepared as follows:

The dried onions, also called onion chips, can be extracted, for example, with purified water, e.g. in the ratio of drug to extractant (water) of 1:16. The ratio of dry drug to native extract-then corresponds to 1.8:1, or 1.5-2.2:1. The ratio of dry drug to fluid extract is then 0.16:1. The drug (e.g. 16 kg of chips) is exhaustively percolated with hot water (80-90 degrees Celsius). The percolate can then be evaporated at about 55 degrees Celsius under reduced pressure to give the spissum extract (thickened extract). During this, heating is briefly carried out (e.g. 3 seconds at 141 degrees Celsius). The resulting spissum extract is thick-liquid and is dissolved in water, alcohol or in the water-alcohol mixture to give the fluid extract e.g. of the abovementioned ratio. This gives a reddish-brown to brown liquid with a characteristic odor. This extract is miscible with water in any ratio, has a relative density at 20 degrees Celsius of 1.00-1.03 g/ml. If water/alcohol, in particular ethanol, has been used for the processing, approximately 13-20% (v/v) of alcohol (e.g. ethanol) may be present. The dry residue (after 2 hours at 105 degrees Celsius) is at least 7.0% (m/m). With regard to the microbiological purity, the product corresponds to the requirements of category 3 of German Pharmacopeia 10.

The characteristic ingredients of the onions are derivatives of sulfur-containing amino acids. Protein, fat and carbohydrates are also present.

The product prepared as described above is obtainable, for example, from Finzelberg Extrakte and described in the abovementioned U.S. Pat. No. 5,885,581.

This extract obtained as described above is used in the scar gel according to U.S. Pat. No. 5,885,581.

In contrast to the gel composition, however, it was surprising to incorporate this extract into an oil-containing or oil-water-containing base whilst at the same time continuing to ensure complete and homogeneous distribution of the active ingredients and also in particular their remaining intact, especially when the proportion of this extract phase, in particular in aqueous, alcoholic, aqueous/alcoholic form, is very high.

Preference is thus given to using a water, alcohol onion extract in the agents according to the invention, in particular a water-ethanol extract as described above, preferably with 10-40%, in particular 13-20%, in particular 10-15% ethanol content.

As mentioned, the onion extract can, instead of ethanol, also have other solvents, in particular glycol, butylene glycol, propylene glycol.

Also preferred is an alcohol-, preferably ethanol-containing, extract which, in particular, can moreover, as mentioned, also have further solvents, e.g. 10-80%, in particular 20-60%, such as those specified under point II. Preference is given here to hydrocarbons, triglycerides, e.g. medium-chain ones, fatty acid esters.

Also suitable are mixtures of a water/alcohol, in particular ethanol-onion extract, e.g. with 10-40% ethanol, as described above and an alcoholic, in particular ethanolic, onion extract with 10-80%, in particular 20-60%, solvent, such as those specified under point II, in particular here hydrocarbons, triglycerides e.g. medium-chain ones, fatty acid esters. The ratio of water/alcohol and alcohol/solvent onion extract can preferably be, in particular, 3:1 to 1:3, preferably 1:1.

II. Fat (Oil) Phase

For the fat (oil) phase, oil components customary for this can be used. These include:

Customary, preferably liquid, lipids, these may be present individually or in mixture.

In particular, the following groups and examples are suitable for this:

### 1. Hydrocarbons

Hydrocarbons, such as squalene, squalane, in particular also liquid paraffins (Paraffinum perliquidum), isoparaffins, dioctylcyclohexanes (Cetiol® S), isohexadecanes (Arlamol® HD);

2. Fatty alcohols such as oleyl alcohol, octyldodecanol (Eutanol® G);
3. Fatty acid esters, e.g. isopropyl fatty acid esters (palmitate, myristate, isostearate, oleate), decyl oleate (Cetiol® V), hexyl laurate, C12-15 alkyl benzoates (Finsolv® TN), dicaprylyl carbonate (Cetiol® CC), diesters, such as dibutyl adipate (Cetiol® B), propylene glycol dipelargonate, branched fatty acid esters, such as PCL-liquid® (cetearyl octanoate) or mixtures such as Cetiol® PGL (hexyldecanol and hexyldecyl laurate);
4. Fatty alcohol ethers such as dicaprylyl ether (Cetiol® OE) or fatty alcohol esters such as lactic eaters, such as C12-13 alkyl lactates (Cosmacol® ELI);
5. Polyol fatty acid esters such as Cetiol® HE (PEG-7 glyceryl cocoate);
6. Trialycerides, in particular medium-chain ones (neutral oils), such as caprylic/capric triglycerides (Miglyol® 810, 812) and polyol esters thereof, such as propylene glycol dicaprylate/dicaprate (Miglyol® 840);
7. Natural fats and oils, such as sunflower oil, soybean oil, peach kernel oil, apricot kernel oil, grapeseed oil, castor oil, peanut oil, almond oil, mink oil, wheatgerm oil, avocado oil, evening primrose oil;
8. Waxes, such as natural liquid waxes, e.g. jojoba oil or its substitute oleyl erucate (Cetiol® J 600) or synthetic waxes, such as those described below under “consistency-imparting agents”.
9. Silicone oils and waxes, e.g. polydimethylsiloxanes, such as Dow Corning Fluid® 200 (dimethicone), cyclomethylsiloxane, such as Dow Corning Fluid® 345 (cyclomethicone), phenylmethylpolysiloxane, such as phenyl dimethicone (Abil® AV 8853) or alkylpolymethylsiloxane copolymers, such as cetyl dimethicone (Abil® Wax 9801), stearyl dimethicone (Abil® Wax 9800), dialkoxydimethylpolysiloxane, such as stearoxy dimethicone (Abil® Wax 2434), behenoxy dimethicone (Abil® Wax 2440).

Particularly preferred oil components are liquid paraffins, fatty acid esters, such as isopropyl palmitate or myristate, fatty alcohol ethers, such as dicaprylyl ether (Cetiol® OE), and said natural fats and oils, in particular avocado oil, soybean oil, peach kernel oil, apricot kernel oil and very particularly evening primrose oil, in particular mixtures thereof, silicone oils of the above described type, and lactic eaters, e.g. Cosmacol® ELI, and mixtures thereof with the abovementioned components.

Of particular preference are said liquid paraffins, lactic esters, fatty alcohol ethers, evening primrose oil and silicone oils, also in combination with one another, where about 1-50% of individual components, based on the total amount of oil, may be present.

Silicone waxes are also particularly suitable, especially also combinations thereof and also with the abovementioned paraffins, lactic esters and evening primrose oil.

### III. Emulsifiers

### A). W/O Emulsifiers

Suitable W/O emulsifier(s) are those with a suitable HLB value of 1-9, in particular 1-8. Particular preference is given to the following products:

### 1. Sorbitan Derivatives

sorbitan esters, such as sorbitan oleate (Span® 80, HLB=4.5), sorbitan stearate (HLB=5.0), sorbitan sesquioleate (Crill® 43, HLB=3.7), sorbitan isostearate (Crill® 6, HLB=4.7), sorbitan tristearate (Crill® 35, HLB=2.1); and Arlacel® 582 (sorbitan isostearate, PEG-2 hydrogenated castor oil, ozokerite, hydrogenated castor oil, HLB=5).

### 2. Polyethoxylated Products

polyethoxylated fatty acids and fatty alcohols, such as PEG-2 oleate (HLB=5.0), PEG-4 distearate (HLB=3.0), PEG-2 stearate (HLB=4.4), ceteareth-3, (Volpo® CS3, HLB=5.0), ceteth-2 (Volpo® C2, HLB=5.3); polyoxyethylene fatty acid esters, such as Arlatone® T(V) (PEG-40 sorbitan peroleate, HLB=9); ethoxylated triglycerides, such as PEG-5 castor oil (HLB=3.9), PEG-6 diricinoleate (HLB=5.0), PEG-7 hydrogenated castor oil (Cremophor® WO 7, HLB=5.0);

### 3. (Poly)Glyceryl Derivatives

polyglyceryl esters such as polyglyceryl-3 diisostearate (Lameform® TGI, HLB=3.5), polyglyceryl-2 dipolyhydroxystearate (Dehymuls® PGPH, HLB=3.5), diisostearoyl polyglyceryl-3 diisostearate (Isolan® PDI, HLB about 5), polyglyceryl-3 oleate (Isolan® GO 33, HLB about 5), polyglyceryl-3 dioleate (Cremophor® GO 32, HLB about 5), polyglyceryl-4 isostearate (Isolan® GI 34, HLB about 5); glycerol esters, such as glyceryl ricinoleate (Cithrol® GMR N/E, HLB=2.7), glyceryl laurate (Cithrol® GML N/E, HLB=4.9), glyceryl dioleate S/E (Cithrol® GDO S/E, HLB=2.9).

### 4. Polyol Esters

polyol esters, such as glycol oleate S/E (Cithrol® EGMO S/E, HLB=2.7), glycol ricinoleate (Cithrol® EGMR S/E, HLB=2.0), glycol dilaurate S/E (Cithrol® EGDL S/E, HLB=2.0), propylene glycol ricinoleate (Cithrol® PG MR S/E, HLB=3.6), propylene glycol laurate (Cithrol® PGML N/E, HLB=2.7);

### 5. Glucose Derivatives

glucose esters, such as methyl glucose dioleate (Isolan® DO, HLB about 5), methyl glucose isostearate (Isolan® IS, HLB about 5);

### 6. Pentaerythritol Derivatives

pentaerythritol fatty acid esters, e.g. pentaerythrityl monolaurate (HLB=4.8), pentaerythrityl monotallate (HLB=4.0) or mixed esters, e.g. with citric acid, fatty alcohol esters, such as Dehymuls® E (dicocoyl pentaerythrityl distearyl citrate, sorbitan sesquioleate, Cera alba, aluminum stearate, HLB=4.0), Dehymuls® F (dicocoyl pentaerythrityl distearyl citrate, Cera microcristallina, glyceryl oleate, aluminum stearate, propylene glycol, HLB=4.0);

### 7. Alkylphenols

e.g. nonoxynol-2 (HLB about 4.5);

### 8. Polymers

polymers, such as polyoxypropylene-polyoxyethylene block polymers (INCI name: Poloxamers), e.g. Pluronic® PE 3100 (HLB=4.5), Pluronic® PE 6100 (HLB=3.0) or PEG-30 dipolyhydroxystearate (Arlacel® P 135, HLB about 5.5);

### 9. Siloxane Derivatives

polysiloxane copolymers, such as polysiloxane-polyether copolymers, in particular polysiloxane-polyalkyl-polyether copolymers, such as cetyl dimethicone copolyol (Abil® EM 90, HLB=about 5), laurylmethicone copolyol (Dow Corning® Q2-5200, HLB about 4) or mixtures thereof, such as Abil® WE 09 (cetyl dimethicone copolyol polyglyceryl-4 isostearate, hexyl laurate, HLB about 5).

### 10. Fatty Acid Salts

polyvalent salts, such as magnesium stearate, aluminum stearate or zinc stearate, preference being given to magnesium stearate.

Particularly preferred emulsifiers are Abil® EM 90 (cetyl dimethicone copolyol) or Arlacel® 582 (sorbitan isostearate, PEG-2 hydrogenated castor oil, ozokerite, hydrogenated castor oil, HLB=5) or magnesium stearate, aluminum stearate, zinc stearate, in particular mixtures thereof. In addition, said emulsifiers can also be combined with suitable coemulsifiers to give the desired HLB value. These include, for example, Arlatone® T(V), in particular in combination with the preferred W/O emulsifiers.

### B) O/W Emulsifiers

Suitable O/W emulsifiers are, in particular, those with a HLB value of 9-18, preferably 9-15 and in particular 9-13.

These preferably include polyoxyethylated products such as (HLB values on the right hand side):

| G-2111 | polyoxyethylene oxypropylene oleate | 9.0 |
|---|---|---|
| G-2125 | tetraethylene glycol monolaurate | 9.4 |
| Brij ® 30 | polyoxyethylene lauryl ether | 9.5 |
| Tween ® 61 | polyoxyethylene sorbitan | 9.6 |
| | monostearate | |
| Tween ® 81 | polyoxyethylene sorbitan monooleate | 10.0 |
| G-3806 | polyoxyethylene cetyl ether | 10.3 |
| Tween ® 65 | polyoxyethylene sorbitan | 10.5 |
| | tristearate | |
| Tween ® 85 | polyoxyethylene sorbitan trioleate | 11.0 |
| G-3910 | polyoxyethylene oleyl ether | 12.2 |
| G-2127 | polyoxyethylene monolaurate | 12.8 |
| Renex ® 690 | polyoxyethylene alkyl aryl ether | 13.0 |
| | polyethylene glycol-400 monolaurate | 13.1 |
| Cremophor ® | polyoxyethylene castor oil | 13.3 |
| EL | | |
| G-1284 | polyoxyethylene castor oil | 13.3 |
| Tween ® 21 | polyoxyethylene sorbitan | 13.3 |
| | monolaurate | |
| Renex ® 20 | polyoxyethylene esters of mixed | 13.5 |
| | fatty acids and resin acids | |
| G-1441 | polyoxyethylene sorbitol lanolin | 14.0 |
| | derivative | |
| G-7596J | polyoxyethylene sorbitan | 14.9 |
| | monolaurate | |
| Tween ® 60 | polyoxyethylene sorbitan | 14.9 |
| | monostearate | |
| Tween ® 80 | polyoxyethylene sorbitan monooleate | 15.0 |
| Myrj ® 49 | polyoxyethylene sorbitan | 15.0 |
| | monostearate | |
| G-3720 | polyoxyethylene stearyl ether | 15.3 |
| G-3920 | polyoxyethylene oleyl ether | 15.3 |
| Tween ® 40 | polyoxyethylene sorbitan | 15.6 |
| | monopalmitate | |
| G-2162 | polyoxyethylene oxypropylene | 15.7 |
| | monostearate | |
| Cremophor ® 0 | polyoxyethylene fatty alcohol ether | 16.0 |
| G-1471 | polyoxyethylene sorbitol lanolin | 16.0 |
| | derivative | |
| Myrj ® 51 | polyoxyethylene monostearate | 16.0 |
| Cetomacrogol | polyoxyethylene glycol-1000 | 16.1 |
| 1000 | monocetyl ether | |
| Tween ® 20 | polyoxyethylene sorbitan | 16.7 |
| | monolaurate | |
| Brij ® 35 | polyoxyethylene lauryl ether | 16.9 |
| Myrj ® 52 | polyoxyethylene monostearate | 16.9 |
| Myrj ® 53 | polyoxyethylene monostearate | 17.9 |
| In addition, preference is also given to: | | |
| Myrj ® 45 | polyoxyethylene glycol monostearate | 11.2 |
| | polyoxyethylene glycol-400 | 11.4 |
| | monooleate | |
| Cremophor ® | polyoxyethylene glycol-400 | 11.6 |
| AP-solid | monostearate | |
| G-2161 | polyoxyethylene glycol-400 | 11.6 |
| | monostearate | |
| Brij ® 721 P | steareth-21 | 15.5 |
| Eumulgin ® B1 | ceteareth-12 | 13 |

Preference is also given to standard commercial mixtures with coemulsifiers, such as fatty alcohols or glyceryl esters, such as emulsifier E 2149 (steareth-7, stearyl alcohol, HLB=11) or Arlacel® 165 FL (glyceryl stearate, PEG-100 stearate, HLB=11) and also

| | | |
|---|---|---|
| Atlox ® 3300 | alkylarylsulfonate | 11.7 |
| | triethanolamioleate | |

Preference is also given to nonionic and ionic phosphates, such as cetyl or stearyl phosphates (HLB=8, or 10), trilaureth-4 phosphate (Hostaphat® KL 340, HLB about 13), triceteareth-4 phosphate (Hostaphat® KW 340, HLB=10), (poly)glyceryl esters, such as Dermofeel®Q 182 S (polyglyceryl-10 distearate) or PEG-4 polyglyceryl-2 stearate (Hostacerin® DGSB, HLB about 8), polyglyceryl-2 PEG-10 laurate (Hostacerin® DGL, HLB about 14), polyglyceryl-3 methyl glucose distearate (Tego Care® 450, HLB=12) or mixtures, such as Eumulgin® VL 75 (lauryl glucoside, polyglyceryl-2 dipolyhydroxystearate, glycerol, HLB about 13). Preference is also given to sugar esters, such as glucose esters, e.g. cetearyl glucoside (Tego Care® CG 90, HLB=11), methyl glucose sesquistearate (Tego Care® PS, HLB=12), PEG-20 methyl glucose sesquistearate (Glucamate® SSE, HLB=15) or mixtures with fatty alcohols, such as Montano® 82 (cetearyl alcohol, cocoglucoside, HLB=11), Montanov® 14 (myristyl alcohol, myristyl glucoside, HLB=10), Montanov® 202 (arachidyl alcohol, behenyl alcohol, arachidyl glucoside, HLB=8) or sucrose esters, such as sucrose stearate (Crodesta® F 160, HLB=14.5), sucrose cocoate (Crodesta® SL 40, HLB=15) or mixtures thereof, e.g. Crodesta® F 110 (sucrose stearate, sucrose distearate, HLB=12).

In addition, sterol derivatives can be used as emulsifiers, e.g. lanolin derivatives, such as laneth-20 (Polychol® 20, HLB=14) or Generol® RE 10 (PEG-10 rapeseed sterol, HLB=12) or castor oil derivatives such as PEG-40 hydrogenated castor oil (Eumulgin® HRE 40, HLB=15) or PEG-36 castor oil (Arlatone® 650, HLB=12.5), or mixtures thereof, such as Arlatone® 980 (PEG-15 hydroxystearate, PEG-25 hydrogenated castor oil, HLB=12.8).

Siloxane derivatives can also preferably be used, in particular polysiloxane-polyether copolymers, such as Abil® Care 85 (dimethicone copolyol, caprylic/capric triglyceride, HLB=10).

Ionic emulsifiers which can be used here are monovalent salts e.g. of fatty acids or fatty alcohol sulfates, e.g. sodium stearate or triethanolaminostearate, Lanette® E (sodium cetearyl sulfate) or else phosphates, such as Amphisol® K (potassium cetyl phosphate) or glutamates, e.g. Hostapon® CCG (sodium cocoyl glutamate) or lactylates, e.g. Crolactil® SSL (sodium stearoyl lactylate).

All of the abovementioned emulsifiers can also be used in mixtures with coemulsifiers, which can also generally be used as consistency-imparting agents. These may be chosen from the group of fatty alcohols, such as stearyl alcohol (Lanette® 18), cetyl alcohol (Lanette® 16), myristyl alcohol (Lanette® 14) or cetearyl alcohol (Lanette® O). Also suitable are fatty acids, e.g. stearic acid or glyceryl esters, such as glyceryl stearate, in particular glycerol monostearate or glycerol distearate or mixtures thereof, e.g. Tegin® M.

Consistency-imparting agents which can also be used are waxes, e.g. beeswax (Lunacera® alba), Kester® wax K82H (C₂₀₋₄₀-alkyl stearate) or Lunacera® M (microwax) or hydrocarbon waxes, such as Lunacera® P (mineral wax), and hydrogenated castor oil (Cutina® HR) or synthetic waxes, such as cetyl palmitate (Cutina® CP) or myristyl myristate (Crodamol® MM), or stearyl stearate (Crodamol® SS).

Particularly preferred O/W emulsifiers are:

Tego Care® 450 (polyglyceryl-3 methyl glucose distearate, HLB=12),

Eumulgin® 1 (ceteareth-12, HLB=13) or mixtures thereof.

Also preferred are the abovementioned Hostaphat® products or Abil® Care 85.

### IV. Additives

### 1) Additional Active Ingredients

Additional active ingredients which may be chosen are, in particular, vitamins, such as, for example, tocopherol acetate (vitamin E) or vitamin A, e.g. as retinol palmitate. Also suitable are electrolytes, such as magnesium sulfate or sodium chloride (electrolytes e.g. in amounts of 0.2-2%). It is also possible to use polysaccharides, such as glycosamine glycans, in particular mucopolysaccharides. These include in particular non-heparinoid compounds, such as, for example, chondroitin sulfate or dermatan sulfate or keratan sulfate or else heparinoid compounds, such as heparin, in particular salts thereof, e.g. sodium salts.

Also suitable are, in particular, allantoin, D-panthenol, hyaluronic acid and/or zinc derivatives, such as Zincidone® (zinc PCA), zinc gluconate or zinc oxide.

The amounts of individual active ingredients vary and can, for example, be from in each case 0.01-20% or 0.1-6%, in particular 1-5% or 3-5%.

Moreover, vesicle formers, in particular lecithins and analogs thereof are suitable as additional active ingredients. These include, for example, known substances (cf. DE 42 05 548 C2), in particular phospholipids, such as lecithin (egg or soybean lecithin), e.g. Phosal® 50 SA (about 50% soybean lecithin), phosphatidylcholine, -serine or -diethanolamine, and mixtures thereof.

Further suitable lecithin-analogous components are sphingolipids (e.g. ceramides, cerebrosides, sphingosine, sphingomyelin), phytosterols (essentially mixtures of β-sitosterol, campesterol and stigmasterol), and derivatives thereof, in particular ethoxylates, such as Generol® 122 E 5 (PEG-5 soybean sterol), Gererol® R E5 (PEG-5 rapeseed sterol).

Further suitable vesicle formers are polyethoxylated fatty alcohols, and fatty acids having preferably 1-4 EO with a HLB value of 2 to 6, where the lipophilic radical preferably consists of C₁₆ to C₁₈-alkyl chains, polyglycerol alkyl ethers, glucosyl dialkyl ethers, sucrose diesters, collagen hydrolyzate esters, quaternary ammonium compounds and poloxamers. Particular preference is given to egg and/or soybean lecithin (Phosal®50SA), phosphatidylcholine, ceramides, phytosterols and ethoxylated derivatives thereof with a degree of ethoxylation of from 5 to 16 and polyoxyethylated fatty alcohols with a HLB value of 2-6 or combinations thereof, in particular of phytosterols of the abovementioned type with ethoxylation products thereof. Particular preference is given here to Phosal® 50 SA.

Particularly preferred additional active ingredients are:
a) vitamin E (e.g. 0.1-5%, in particular 0.1-1%);
b) magnesium sulfate and/or sodium chloride (e.g. 0.1-5%, in particular 0.1-1%);
c) mucopolysaccharides, such as, for example, chondroitin sulfate (e.g. 0.1-2%, in particular 0.1-1%), and heparin or heparinoids, such as heparin-Na (e.g. 0.1-2%, in particular 0.1-1%);
d) allantoin; amounts as above; e.g. 0.05-2%, preferably 0.1-1%;
e) D-panthenol; amounts as above; e.g. 0.1-10%, preferably 1-5%;
f) hyaluronic acid (e.g. 0.001-1%, in particular 0.01-0.1%);
g) lecithins, such as Phosal® 50 SA (e.g. 0.5-5%, in particular 1-2%);
h) zinc derivatives, such as zinc gluconate or zinc PCA (Zincidone®), e.g. 0.1-3%, in particular 0.5-1%
i) oil-absorbing substances, e.g. starch derivatives, such as Natrasorb® HFB (aluminum starch, octenyl succinate, acrylate copolymer, magnesium carbonate) or acrylates, e.g. Micropearl® M 100 (polymethyl methacrylate), Micropearl® M 305 methyl methacrylate copolymer) or sulfonates, such as Biopol® OE (sodium C8-16 isoalkylsuccinyl lactoglobulin sulfonate).

It is also possible to use astringent and sebum-regulating substances, such as Acnacidol® 101 (propylene glycol, hydroxydecanoic acid), Asebiol® BT (hydrolyzed yeast protein, pyridoxine, niacinamide, glycerol, panthenol, propylene glycol, allantoin, biotin), Lipacide® C8C0 (caproyl collagen aminoacids), Sebosoft® (glycerol, aqua, PEG-8, caprylyl glycol, sebacic acid, sodium polyacrylate), Sepi Control® A5 (capryloylglycine, methyiglycine, Cinnamonum zeylanicum).

Further additional active ingredients which may be present are further plant extracts, e.g. birch leaf extract, aloe vera extract, marigold extract, hibiscus extract, burdock extract, hamamelis extract, gotu kola extract, algae extract, quince extract, water lily extract, cinnamon extract.

It is also possible to incorporate cooling/calming active ingredients, such as Frescolat® ML (menthyl lactate) or Eashave® (sodium hyaluronate, wheat germ extract, Saccharomyces cerevisiae extract).

Moreover, additional active ingredients which may be used are circulation-promoting substances, e.g. nicotinic acid derivatives, such as methyl or tocopheryl nicotinate, alpha- and beta-hydroxyacids and derivatives thereof, e.g. glycolic acid, malic acid, citric acid, tartaric acid, lactic acid, salicylic acid, isopropylbenzyl salicylates, C12-13 alkyl lactates (Cosmacol® ELI) or also antiphlogistic and antibacterial substances, such as triterpenes, e.g. ursolic acid, glycyrrhicinic acid or glycyrrhetinic acid and derivatives thereof, e.g. stearyl glycyrrhetinate, potassium glycyrrhinate; pantothenic acid derivatives, e.g. D-panthenol, panthenyl triacetate; allantoin; bisabolol; azulenes, e.g. chamazulenes or guaiazulene; phytosphingosines; triclosan; chlorhexidine derivatives and/or antidandruff agents, e.g. climbazole or piroctone olamine.

In addition, in the compositions according to the invention it is also possible to use substances with an antioxidative and cell-protective effect, such as flavonoids, e.g. rutin, ferulic acid and esters thereof or isoflavones, such as soybean isoflavones or coenzyme Q10, as effective additives.

Very particular preference is given to additional active ingredient combinations of a)-f) or a)-i) or a)+c)-i). These can then be combined in a suitable manner with the customary additives described below.

### 2) Customary Additives

These are preferably chosen from: antioxidants, perfume substances, dyes, UV filters, preservatives and/or humectants, stabilizers, consistency-imparting agents.

The antioxidants can preferably be chosen from butylhydroxytoluene, butyihydroxyanisole, ascorbyl palmitate, tocopherol, possibly in combination with synergistic agents, such as in Controx® VP (tocopherol, lecithin, ascorbyl palmitate, hydrogenated palm glycerides citrate), gallic alkyl esters, such as octyl, dodecyl and cetyl gallate or combinations thereof.

Perfume substances are, in particular, chosen from essential oils. In addition, standard commercial perfume compositions are also possible, such as, for example, Deliana perfume oil. Essential oils which are suitable for the agents according to the invention are, in particular the essential oils chosen from rosemary oil, orange oil, lavender oil, lime oil, cinnamon oil, geranium oil, cedarwood oil, rosewood oil, valerian oil, ylang-ylang oil, citronella oil, tea tree oil, manuca oil, eucalyptus oil, mint oil, lemongrass oil, cypress oil, niaouli oil, spruce needle oil, pine needle oil, camphor, menthol.

These additives can also be regarded as additional active ingredients. These then also include essential unsaturated fatty acids and esters thereof, e.g. linoleic acid or linolenic acid, glyceryl linoleate, glyceryl linolenate are used.

Particularly preferred perfume or additional active ingredients are chosen from essential oils, plant extracts and plant oils, sebum-regulating substances, humectants, antiphlogistic and antibacterial substances, vitamins, unsaturated essential fatty acids or mixtures thereof.

Preferred dyes are, for example, Patent Blue, amido blue, orange RGL, cochineal red, dye FD+C Blue No. 1 or titanium dioxide or quinoline yellow.

Suitable UV filters are UVB, UVA and broadband filters of the following type:

UV-B filters: cinnamic esters, e.g. octyl methoxycinnamate (Eusolex® 2292, Neo Heliopan® AV, Parsol® MCX), isoamyl p-methoxycinnamate (Neo Heliopan® Galanga) and 4-methylbenzylidenecamphor (Euaolex® 6300), paraaminobenzoic acid and esters, such as 2-ethylhexyl N,N-dimethyl-4-aminobenzoate (Eusolex® 6007, octyldimethyl PABA), homomenthyl salicylate (Homosalate, Eusolex® HMS), octylsalicylate (Neo Heliopan® OS), octocrylene (Neo Heliopan® 303), phenylbenzimidazolesulfonic acid (Neo Heliopan® Hydro, Eusolex® 232)

UVA+UVB filters for broadband absorption, such as benzophenone-3 (Neo® Heliopan BB, Eusolex® 4360);

UV-A filters, such as methyl anthranilate (Neo Heliopan® MA), butylmethoxydibenzoylmethane (Parsol® 1789, Eusolex® 9020); methylene bis-benzotriazolyltetramethylbutylphenol (Tinosorb® M), bis-ethylhexyloxyphenolmethoxyphenyltriazine (Tinosorb® S), disodium phenyldibenzimidazole tetrasulfonate (Neo Heliopan® AP).

Particular preference is given to octyl or isoamyl p-methoxycinnamate, octocrylene, 4-methylbenzylidenecamphor, homosalate and/or butyl methoxydibenzoylmethane and/or benzophenone-3.

Also suitable are inorganic UV filters, such as zinc oxide and titanium dioxide, in particular micronized and/or coated, e.g. Z-cote®, Tioveil®.

Suitable preservatives are iodopropynylbutylcarbamate, DMDM hydantoin, phenoxyethanol and further customary preservatives, such as, for example, sorbic acid and dehydracetic acid and salts thereof, methyldibromoglutanonitrile, etc. or combinations thereof, or other acids, such as benzoic acid or salicylic acid, or benzyl alcohol or esters, such as p-hydroxybenzoic esters, e.g. methyl-, ethyl-, propyl-, butyl-, isobutylparaben, preferably methyl- or propylparaben or mixtures thereof or climbazole or suitable combinations of said substances, such as, for example, methyl-, propylparaben and sorbic acid. Particularly suitable are mixtures, e.g. of DMDM hydantoin and iodopropynylbutylcarbamate such as Glydant® plus.

Also suitable are humectants, e.g. polyalcohols, such as polyethylene glycol, propylene glycol, butylene glycol, sorbitol, glycerol or polymers, e.g. polyquaternium grades, such as polyquaternium-39 (Merquat® plus 3330), proteins, such as collagen or hydrolysates thereof, amino acids, urea, D-panthenol, plant proteins, such as, for example, from wheat, soybean or almond or hydrolysates thereof, e.g. Tritisol® (hydrolyzed wheat protein), polysaccharides, such as, for example, Fucogel® 1000 (biosaccharide gum-1), glucosaminoglycans, e.g. hyaluronic acid or sulfated glucosaminoglycans such as chondroitin sulfate, dermatan sulfate, keratan sulfate, heparan sulfate, in particular their Na salts or heparin-Na, glucans, e.g. β-glucan, e.g. oat β-glucan (Drago-β-Glucan®), mannans, such as konjac mannans, algae extracts, e.g. Seamannin® SU or standard commercial humectants, such as, for example, Hydractin® (glycerol, aqua, disodium adenosine triphosphate, algin, Carica papaya) or Aquaderm® (sodium PCA, sodium lactate, fructose, glycine, niacinamide, urea, inositol), salts, e.g. sodium lactate, DL-2-pyrrolidone-5-carboxylic acid, Na salt.

Preference is given to polyethylene/propylene glycol or glycerol in amounts of, for example, 0.5-10%, in particular 2-5%, and polysaccharide compounds, such as Fucogel® 1000.

Suitable stabilizers are firstly wax products, such as, for example, Lunacera® alba (Cera alba), Lunacera® M (Cera microcristallina), Cutina® HR (hydrogenated castor oil) or silicone waxes, e.g. Abil® wax 9800 (stearyl dimethicone). It is also possible to use Amerchol® CAB consisting of petroleum jelly and lanolin, as stabilizers.

Secondly, stabilizers which can be used are components for regulating the pH, such as NaOH (e.g. 5% strength, e.g. in amounts of 0.1-4%, in particular 1-3%) or acids, such as e.g. citric acid, lactic acid or malic acid or EDTA Na in a suitable amount as complexing agent. The amount and type of pH regulators depends on the other additives and are known to the person skilled in the art.

In particular a suitable stabilizer is a combination of one or more acrylamides, one or more acrylates and one or more polysaccharides, in particular starch, or starch derivatives, where each component may be present in an amount of 0.05-8%, preferably 0.1-5%.

Particularly preferred acrylamides are polyacrylamide, e.g. Flocare® T 920 GC or polyacrylamide-containing mixtures, such as Sepigel® 305 (polyacrylamide, C13-14 isoparaffin, laureth-7), Sepigel® 501 (acrylamides copolymer, mineral oil, C13-14 isoparaffin, polysorbate 85), Sepigel® 502 (C13-14 isoparaffin, isostearyl isostearate, sodium polyacrylate, polyacrylamide, polysorbate 20), Creagel® EZ DC (polyacrylamide, polydecene, dimethicone copolyol), Creagel® EZ 5 (polyacrylamide, polydecene, laureth-5).

Acrylates which can be used are carboxyvinyl copolymers with a high molecular weight (1-3 million), and copolymers thereof, in particular following neutralization by an alkali. Preference is given to the Carbopol® grades known under the INCI name Carbomer, e.g. Carbopol® 910, 934, 940, 941, 954, 980, 981, 2984, 5984 or Carbopols ETD 2001, 2050 or Synthalen® K, L, M or the already neutralized carbomers, such as PNC® 400, 410, 430 (INCI: Sodium Carbomer). It is also possible to use acrylate copolymers, e.g. acrylates/C10-30 alkyl acrylate crosspolymer, known as Carbopol® 1342, 1382, ETD 2020, Pemulen® TR-1, TR-2.

Suitable starch or starch derivatives are, in particular, the following substances: rice starch, wheat starch, corn starch and potato starch.

Particular preference is given to hydrophobically modified starches, such as aluminum starch octenylsuccinate (Dry Flo® PC, Fluidamid® DF 12) or mixtures thereof, such as Natrasorb® HFB (aluminum starch ocetenylsuccinate, acrylate copolymer, magnesium carbonate), ASO/MM3® (aluminum starch octenylsuccinate, magnesium myristate), Dry Flo® Elite LL (aluminum starch octenylsuccinate, lauroyl lysine), Facemat® (aluminum starch octenylsuccinate), mica, Zea mays (corn) starch, silica, titanium dioxide, zinc oxide). Very particular preference is given to Dry Flo® PC and Natrasorb® HFB.

The abovementioned combination of stabilizers can also be used in particular with the use of O/W emulsifiers. Particular preference here is given to Sepigel®305 or 501, PNC®410/400 or Carbopol®ETD2020 and Dry Flo®PC or Natrasorb®HFB, in particular combinations thereof.

Preference is given to using preservatives, antioxidants, dyes and perfume substances together with stabilizers and humectants and additional active ingredients optionally in conjunction with pH regulators and additional substances, particularly preferably in combination with the abovementioned additional active ingredients.

The amounts of additives or active ingredients may vary within the given ranges.

Preference is given to additives chosen from additional active ingredients such as vitamins, electrolytes such as magnesium sulfate and sodium chloride, allantoin, D-panthenol, hyaluronic acid, mucopolysaccharides, such as, for example, heparinoids and nonheparinoids, and mixtures thereof, and perfume substances, preservatives and humectants.

These can preferably be combined with waxes and/or lecithins, such as, in particular, Phosal®50SA.

The described agent containing onion extract can be prepared by combining onion extract preferably with additives soluble therein, then preparing the fat phase, where additives soluble therein may preferably be incorporated, and then preparing the water phase, which preferably has additives soluble therein and in particular alcohols of the abovementioned type and amount, and then emulsifying the water phase and the fat phase at temperatures of from 60 to 90° C. together with one or more emulsifiers or mixtures thereof or with coemulsifiers, optionally homogenizing them and, after cooling (e.g. at 20-50° C.), adding the additives, if present, and processing the mixture in a suitable manner, e.g. by homogenization.

Alternatively, the onion extract can be incorporated together with the additives into the fat (oil) phase, where optionally also solvents, in particular alcohols of the abovementioned type and amount, can be added.

As onion extract, the aqueous, alcoholic, alcoholic-solvent-containing or aqueous-alcohol one desired in each case or mixtures thereof, e.g. in the ratio 3:1 to 1:3, e.g. of the last two mentioned, is chosen in the given amounts.

An agent prepared as described above is suitable particularly, for example, as a dermatological composition for the care, treatment or prevention of damaged skin tissue, in particular scarred tissue, or slackened tissue, such as, for example, stretch marks, or else of damaged skin tissue which may have arisen due to cuts, operation wounds, burns or by age-related degeneration. Even after a short time, the damaged skin tissue exhibits an improvement in the scarred or damaged sections and can, as a result of the new oil phase-containing formulation, be kept significantly softer, more supple and more elastic than without this care, skin-regenerating additive.

Thus, the agents can also surprisingly be used for scars after cosmetic operations and also for the treatment of acne scars with exceptional regenerative success.

The invention is illustrated in more detail by reference to the examples below.

In Examples 1-6, an aqueous-ethanolic onion extract, in particular with a content of 13-20% ethanol, was used, and in Examples 7, 8 an alcohol-containing onion extract, namely containing ethanol and medium-chain triglyceride (about 60%), was used. In Examples 9, 10 the effectiveness is demonstrated by reference to medicinal application observations.

### Example 1

| | |
|---|---|
| Aqua purificata | 34.7879 |
| Magnesium sulfate | 0.7000 |
| Arlacel 582 Nena | 8.0000 |
| Arlatone TV | 1.5000 |
| Lunacera alba | 2.0000 |
| Lunacera M | 2.0000 |
| Magnesium stearate | 2.0000 |
| Amerchol CAB | 5.0000 |
| Cetiol OE | 5.0000 |
| Paraffinum perliquidum | 10.0000 |
| Cosmacol ELI | 3.0000 |
| Evening primrose oil | 2.0000 |
| Tocopherol acetate | 0.5500 |
| Oxynex 2004 | 0.0500 |
| Glydant plus | 0.2000 |
| Propylene glycol | 4.0000 |
| D-Panthenol | 5.5000 |
| Allantoin | 1.0000 |
| Hyaluronic acid | 0.0100 |
| Chondroitin sulfate | 0.2000 |
| Onion extract | 10.0000 |
| Dye pigment paste | 2.0000 |
| Dye FD + C Blue No. 1 | 0.0003 |
| Deliana perfume oil | 0.5000 |

### Example 2

| | |
|---|---|
| Aqua purificata | 51.7879 |
| Sodium chloride | 0.5000 |
| Abil EM 90 | 2.5000 |
| Abil Wax 9800 | 1.0000 |
| Jojoba oil | 1.0000 |
| Arlamol HD | 5.0000 |
| Cetiol OE | 4.0000 |
| Lunacera M | 0.6000 |
| Cutina HR Powder | 0.4000 |
| Cosmacol ELI | 3.0000 |
| Evening primrose oil | 2.0000 |
| Tocopherol acetate | 0.5500 |
| Oxynex 2004 | 0.0500 |
| Dow Corning 345 Fluid | 4.0000 |
| Glydant plus | 0.2000 |
| Propylene glycol | 4.0000 |
| D-Panthenol | 5.5000 |
| Allantoin | 1.0000 |
| Phosal SA 50 New | 2.0000 |
| Hyaluronic acid | 0.0100 |
| Chondroitin sulfate | 0.4000 |
| Onion extract | 10.0000 |
| Dye FD + C Blue No. 1 | 0.0002 |
| Deliana perfume oil | 0.5000 |

### Example 3

| | |
|---|---|
| Aqua purificata | 38.7900 |
| Carbopol ETD 2020 | 0.2500 |
| Tego Care 450 | 3.0000 |
| Eumulgin B1 | 2.0000 |
| Lunacera alba | 2.0000 |
| Kester wax K82H | 3.0000 |
| Arlamol HD | 1.5000 |
| Cetiol SB 45 | 2.0000 |
| Finsolv TN | 3.0000 |
| Dow Corning Fluid 345 | 2.0000 |
| Cosmacol ELI | 3.0000 |
| Evening primrose oil | 2.0000 |
| Tocopherol acetate | 0.5500 |
| Oxynex 2004 | 0.0500 |
| Propylene glycol | 4.0000 |
| Glydant plus | 0.2000 |
| NaOH 5% strength | 2.8000 |
| Trilon BD | 0.1000 |
| Panthenol | 5.5000 |
| Hyaluronic acid Na salt | 0.0100 |
| Phosal SA 50 | 2.0000 |
| Allantoin | 1.0000 |
| Zincidone | 0.5000 |
| Fucogel 1000 | 5.0000 |
| Natrasorb HFB | 3.0000 |
| Onion fluid extract | 10.0000 |
| Dye pigment paste white | 2.0000 |
| Sepigel 305 | 0.5000 |
| Deliana perfume oil | 0.5000 |

### Example 4

| | |
|---|---|
| Aqua purificata | 33.5900 |
| Tego Care 450 | 3.0000 |
| Eumulgin B1 | 2.0000 |
| Lunacera alba | 1.0000 |
| Kester wax K82H | 3.0000 |
| Arlamol HD | 2.5000 |
| Cetiol SB 45 | 2.0000 |
| Finsolv TN | 3.0000 |
| Dow Corning Fluid 345 | 2.0000 |
| Cosmacol ELI | 3.0000 |
| Evening primrose oil | 2.0000 |
| Tocopherol acetate | 0.5500 |
| Oxynex 2004 | 0.0500 |
| PNC410 | 0.2000 |
| Propylene glycol | 4.0000 |
| Glydant plus | 0.2000 |
| NaOH 5% strength | 0.9000 |
| Panthenol | 5.5000 |
| Hyaluronic acid Na salt | 0.0100 |
| Phosal SA 50 | 2.0000 |
| Allantoin | 1.0000 |
| Zincidone | 0.5000 |
| Fucogel 1000 | 5.0000 |
| Sepigel305 | 0.5000 |
| Onion fluid extract | 10.0000 |
| Dye pigment paste white | 2.0000 |
| Dry Flow PC | 5.0000 |
| Deliana perfume oil | 0.5000 |

### Example 5

| | |
|---|---|
| Aqua purificata | 41.0900 |
| Carbopol ETD2020 | 0.2000 |
| Dermofeel Q182S | 3.0000 |
| Eumulgin B1 | 2.0000 |
| Lunacera alba | 2.0000 |
| Kester wax K82H | 3.0000 |
| Arlamol HD | 5.0000 |
| Cetiol SB 45 | 2.0000 |
| Finsolv TN | 3.0000 |
| Dow Corning Fluid 345 | 2.0000 |
| Cosmacol ELI | 3.0000 |
| Tocopherol acetate | 0.5500 |
| Oxynex 2004 | 0.0500 |
| Propylene glycol | 4.0000 |
| Glydant plus | 0.2000 |
| NaOH 5% strength | 1.6000 |
| Panthenol | 5.5000 |
| Hyaluronic acid Na salt | 0.0100 |
| Phosal SA 50 | 2.0000 |
| Allantoin | 1.0000 |
| Zincidone | 0.5000 |
| Fucogel 1000 | 5.0000 |
| Sepigel 305 | 1.0000 |
| Onion fluid extract | 5.0000 |
| Dye pigment paste white | 1.0000 |
| Deliana perfume oil | 0.5000 |
| Natrasorb HFB | 3.0000 |

### Example 6

| | |
|---|---|
| Aqua purificata | 46.1900 |
| Carbopol ETD2020 | 0.2000 |
| Dermofeel Q182S | 3.0000 |
| Eumulgin B1 | 2.0000 |
| Lunacera alba | 2.0000 |
| Kester wax K82H | 1.0000 |
| Arlamol HD | 5.0000 |
| Cetiol SB 45 | 2.0000 |
| Finsolv TN | 3.0000 |
| Dow Corning Fluid 345 | 2.0000 |
| Cosmacol ELI | 3.0000 |
| Tocopherol acetate | 0.5500 |
| Oxynex 2004 | 0.0500 |
| PNC 410 | 0.6000 |
| Propylene glycol | 4.0000 |
| Glydant plus | 0.2000 |
| NaOH 5% strength | 1.6000 |
| Panthenol | 5.5000 |
| Hyaluronic acid Na salt | 0.0100 |
| Phosal SA 50 | 2.0000 |
| Allantoin | 1.0000 |
| Zincidone | 0.5000 |
| Fucogel 1000 | 5.0000 |
| Sepigel 305 | 0.3000 |
| Onion fluid extract | 5.0000 |
| Dye pigment paste white | 1.0000 |
| Deliana perfume oil | 0.5000 |
| Natrasorb HFB | 3.0000 |

### Example 7

| | |
|---|---|
| Miglyol 812 | 73.9000 |
| Onion extract | 2.0000 |
| Tocopherol acetate | 1.1000 |
| Evening primrose oil | 2.0000 |
| Jojoba oil | 5.0000 |
| Cosmacol ELI | 5.0000 |
| Cetiol PGL | 10.0000 |
| Deliana perfume oil | 1.0000 |

### Example 8

| | |
|---|---|
| Miglyol 812 | 86.9000 |
| Onion extract | 2.0000 |
| Tocopherol acetate | 1.1000 |
| Evening primrose oil | 2.0000 |
| Jojoba oil | 3.0000 |
| Cosmacol ELI | 5.0000 |
| Deliana perfume oil | 0.7000 |

### Example 9 Medicinal Application Observations with Patients with a Very Wide Variety of Sears

In 338 patients (62% female) with an average age of 41 years, cuts (45), graze wounds (14), operation wounds (240), burn scars (18), acne scars (6) or other scars (15) in the head/neck region (65), arms (135), legs (84), on the upper body/chest (27), upper body/back (14) or other areas (54) were present over a period of <1 month (106) to 3 months (117) or >3 months. These patients were treated on average twice daily over a period up to 6 months using a product according to the present Example 1 by application to the affected area of skin. Prior to the treatment, in approximately 15% of the cases, a use of known products, such as onion extract gel (contractubex) or ketofibrase had been undertaken without further success. During the treatment according to the invention, an assessment was made with regard to skin smoothing (appearance of the scar like other skin or slightly or considerably hardened, reduction in scar depth, length), skin compatibility (spreadability, absorption, odor, feel on the skin). The results are given in Table 1 below and show that the product according to the invention led to a positive result in more than 90% of cases.

TABLE 1

| | Doctor's | Patient's |
|---|---|---|
| | assessment | assessment |
| Scar smoothing: | | |
| a) very good | 45.0% | 46.7% |
| b) good | 47.6% | 44.7% |
| c) average | 5.6% | 8.0% |
| Overall judgment | 91.4% | 92.6% |
| a) + b) | | |
| Skin | | |
| compatibility: | | |
| a) very good | 75.9% | 70.3% |
| b) good | 23.2% | 28.5% |
| c) average | 0.9% | 1.2% |
| Overall judgment | 99.1% | 98.8% |
| a) + b) | | |

### Example 10: Medicinal Application Observation for the Prevention and Treatment of Stretch Marks

741 subjects were observed from the 16th week of pregnancy with regard to the formation of, prevention of stretch marks over a period of 5-7 months until 8 weeks after the birth, and were treated 1-2 times daily with a product according to Example 2 above on the tummy, breast and upper thigh. At the start of the study 62.5% had no marks, and 37.3% did have marks (of which 19.2% had slight marks, 14.7% had many slight marks, 3.4% had many considerable marks). Of the subjects, 65.8% were primigravidae (of which 7% had marks) and 34.1% were multigravidae (of which 29.1% had marks). After the observation period, it was found that despite an average weight increase of about 12 kg, in the case of 35.9% no stretch marks developed and only in the case of 37.1% did a few slight marks arise, whereas in the case of 21.3% many slight marks were found and only in the case of 5.7% were considerable marks found. The product according to the invention is therefore extremely highly suitable for the effective prevention or reduction of stretch marks, which is clearly evident from the assessment values (criteria analogous to Example 9) given in Table 2.

TABLE 2

| | Doctor's | Patient's |
|---|---|---|
| | assessment | assessment |
| Development of | | |
| marks: | | |
| a) very good | 53.0% | 51.1% |
| b) good | 39.0% | 40.1% |
| c) average | 7.0% | 6.0% |
| d) poor | 1.0% | 2.3% |
| Overall judgment | 92.0% | 91.1% |
| a) + b) | | |
| Skin | | |
| compatibility: | | |
| a) very good | 69.6% | 75.7% |
| b) good | 28.1% | 22.9% |
| c) average | 0.9% | 0.9% |
| Overall judgment | 97.7% | 98.6% |
| a) + b) | | |

1. A method of topically treating, or caring for damaged skin tissue, the method comprising:
producing an oil-in-water agent selected from a lotion, a fluid, a cream or a balsam/ointment comprising
5-15% by weight of an onion extract,
5-40% by weight of a fat (oil) phase, and
0.1-25% by weight of additives selected from the group consisting of consistency-imparting agents, dyes, perfume substances, humectants, antioxidants, preservatives, and stabilizers; and
applying the agent topically to skin to treat, or care for, damaged skin tissue, without phase separation arising; and
wherein the onion extract is selected from a water/ethanol onion extract with 10-40% ethanol or an alcoholic onion extract comprising 10-80% of a solvent chosen from fatty acid esters, triglycerides, hydrocarbons or mixtures thereof.

2. The method as claimed in claim 1, wherein the agent comprises 5-25% by weight of the fat (oil) phase, 0.1-20% by weight of the additives and 5-10% by weight of the onion extract.

3. The method as claimed in claim 1, wherein the agent further comprises an aqueous phase and one or more W/O or O/W emulsifiers or mixtures or mixtures with coemulsifiers.

4. The method as claimed in claim 1, wherein the agent further comprises a first component selected from the group consisting of W/O emulsifiers, O/W emulsifiers, first mixtures of W/O emulsifiers and O/W emulsifiers, and second mixtures of W/O emulsifiers, O/W emulsifiers, and coemulsifiers, and a second component selected from the group consisting of water and a water/alcohol mixture, wherein the first component is present in the amount of 0.1-15%, and wherein the second component is present as a remainder.

5. The method as claimed in claim 1, wherein the agent further comprises a first component selected from the group consisting of at least one W/O emulsifier, first mixtures of at least two W/O emulsifiers, and second mixtures of at least one W/O emulsifier and coemulsifiers, and a second component selected from the group consisting of water and a water/alcohol mixture, wherein the first component is present in the amount of 0.5-15%, and wherein the additives are present in the amount of 0.5-25%, and wherein the second component is present as the remainder.

6. The method as claimed in claim 5, wherein the first component is present in the amount of 0.5-10%, wherein the additives are present in the amount of 0.5-25%, wherein the onion extract is present in the amount of 5-10%, and wherein the second component is present as the remainder.

7. The method as claimed in claim 4, wherein the agent further comprises a first component selected from the group consisting of at least one O/W emulsifier and first mixtures of at least one O/W emulsifier and coemulsifiers, wherein the additives are present in the amount of 0.1-32%, wherein the onion extract is present in the amount of 5-10% and wherein water is present as the remainder.

8. The method as claimed in claim 1, wherein the fat (oil) phase is chosen from hydrocarbons, fatty alcohols, fatty alcohol ethers and esters, (polyol) fatty acid esters, triglycerides, natural oils, natural fats, waxes, silicone oils, silicone waxes or mixtures thereof.

9. The method as claimed in claim 1, wherein the fat (oil) phase comprises liquid paraffins, lactic esters, fatty alcohol ethers, evening primrose oil, silicone oil or mixtures thereof.

10. The method as claimed in claim 2, wherein the agent further comprises a first component selected from the group consisting of W/O emulsifiers and O/W emulsifiers, wherein the W/O emulsifier has a HLB value of 1-8 and the O/W emulsifier has a HLB value of 9-18 or is an ionic O/W emulsifier.

11. The method as claimed in claim 4, wherein the first component comprises a W/O emulsifier comprising sorbitan derivatives, polyethoxylated fatty acids/alcohols/esters/triglycerides, (poly)glyceryl derivatives, polyolesters, glucose derivatives, pentaerythritol derivatives, alkylphenois, (block) polymers, siloxanes, fatty acid salts, or mixtures thereof.

12. The method as claimed in claim 4, wherein the first component comprises a W/O emulsifier comprising cetyl dimethicone copolyol, sorbitan isostearate, polyethylene glycol-2 (PEG-2) hydrogenated castor oil, ozokerite, hydrogenated castor oil, or magnesium stearate, or mixtures thereof.

13. The method as claimed in claim 4, wherein the first component comprises an O/W emulsifier comprising polyoxyethylated products, nonionic and ionic phosphates, ionic monovalent salts, (poly)glyceryl esters, sugar esters, sterol derivatives, castor oil derivatives, siloxanes, or mixtures thereof, or mixtures with coemulsifiers thereof.

14. The method as claimed in claim 13, wherein the agent further comprises an additional O/W emulsifier comprising polyglyceryl-3 methyl glucose distearate, ceteareth-12, or mixtures thereof.

15. The method as claimed in claim 1, wherein the additives comprise stabilizers and a mixture of acrylamides, acrylates and polysaccharides is used as a stabilizer.

16. The method as claimed in claim 1, wherein the additives comprise at least one of vitamins, electrolytes, allantoin, D=panthenol, hyaluronic acid, mucopolysaccharides, dyes, perfume substances, preservatives and humectants.

17. The method as claimed in claim 1, wherein the agent further comprises wax products.

18. The method as claimed in claim 1, wherein the agent further comprises lecithins.

19. The method as claimed in claim 1, wherein the damaged skin tissue is scarred tissue.

20. The method as claimed in claim 19, wherein the scarred tissue is tissue which has arisen as a result of acne or cosmetic operations.

21. The method as claimed in claim 1, wherein the damaged skin tissue is stretch marks.

22. The method as claimed in claim 1, wherein the damaged skin tissue is that which has arisen due to cuts, operation wounds, burns or is damaged skin tissue which has arisen as a result of age-related degeneration.

23. A method of topically treating, or caring for damaged skin tissue, the method comprising:
producing an oil-in-water agent selected from a lotion, a fluid, a cream or a balsam/ointment comprising
5-15% by weight of an onion extract,
5-40% by weight of a fat (oil) phase, and
0.1-25% by weight of additives selected from the group consisting of consistency-imparting agents, dyes, perfume substances, humectants, antioxidants, preservatives, stabilizers, and additional active ingredients; and
applying the agent topically to skin to treat, or care for, damaged skin tissue, without phase separation arising; and
wherein the onion extract is selected from a water/ethanol onion extract with 10-40% ethanol or an alcoholic onion extract comprising 10-80% of a solvent chosen from fatty acid esters, triglycerides, hydrocarbons or mixtures thereof.

24. A method of topically treating, or caring for stretch marks, the method comprising:
producing an oil-in-water agent selected from a lotion, a fluid, a cream or a balsam/ointment comprising
5-15% by weight of an onion extract,
5-40% by weight of a fat (oil) phase, and
0.1-25% by weight of additives selected from the group consisting of consistency-imparting agents, dyes, perfume substances, humectants, antioxidants, preservatives, stabilizers, and additional active ingredients; and
applying the agent topically to skin to treat, or care for, stretch marks, without phase separation arising; and
wherein the onion extract is selected from a water/ethanol onion extract with 10-40% ethanol or an alcoholic onion extract comprising 10-80% of a solvent chosen from fatty acid esters, triglycerides, hydrocarbons or mixtures thereof.

25. A method of topically treating, or caring for scarred tissue, the method comprising:
producing an oil-in-water agent selected from a lotion, a fluid, a cream or a balsam/ointment comprising
5-15% by weight of an onion extract,
5-40% by weight of a fat (oil) phase, and
0.1-25% by weight of additives selected from the group consisting of consistency-imparting agents, dyes, perfume substances, humectants, antioxidants, preservatives, stabilizers, and additional active ingredients; and
applying the agent topically to skin to treat, or care for, scarred tissue, without phase separation arising; and
wherein the onion extract is selected from a water/ethanol onion extract with 10-40% ethanol or an alcoholic onion extract comprising 10-80% of a solvent chosen from fatty acid esters, triglycerides, hydrocarbons or mixtures thereof.

26. A method of topically treating, or caring for stretch marks, or topically treating or caring for scarred tissue, the method comprising:
producing an oil-in-water agent selected from a lotion, a fluid, a cream or a balsam/ointment comprising
5-15% by weight of an onion extract,
5-40% by weight of a fat (oil) phase, and
0.1-25% by weight of additives selected from the group consisting of consistency-imparting agents, dyes, perfume substances, humectants, antioxidants, preservatives, stabilizers, and additional active ingredients; and
applying the agent topically to skin to treat, or care for, stretch marks, or to treat or care for scarred tissue, without phase separation arising; and
wherein the onion extract is selected from a water/ethanol onion extract with 10-40% ethanol or an alcoholic onion extract comprising of 10-80% of a solvent chosen from fatty acid esters, triglycerides, hydrocarbons or mixtures thereof;
wherein the agent further comprises a first component selected from the group consisting of W/O emulsifiers, O/W emulsifiers, first mixtures of W/O emulsifiers and O/W/emulsifiers, and second mixtures of W/O emulsifiers and O/W/emulsifiers, and coemulsifiers, and a second component selected from the group consisting of water and a water/alcohol mixture, wherein the first component is present in the amount of 0.1-15%, and wherein the second component is present as a remainder; and
wherein the first component comprises a W/O emulsifier comprising sorbitan derivatives, polyethoxylated fatty acids/alcohols/esters/triglycerides, (poly)glyceryl derivatives, polyolesters, glucose derivatives, pentaerythritol derivatives, alkylphenols, (block) polymers, siloxanes, fatty acid salts, or mixtures thereof.

27. The method as claimed in claim 26, wherein the first component comprises a W/O emulsifier comprising cetyl dimethicone copolyol, sorbitan isostearate, polyethylene glycol-2 (PEG-2) hydrogenated castor oil, ozokerite, hydrogenated castor oil, or magnesium stearate, or mixtures thereof.

28. The method as claimed in claim 26, wherein the first component comprises a W/O emulsifier comprising polyoxyethylated products, nonionic and ionic phosphates, ionic monovalent salts, (poly) glyceryl esters, sugar esters, sterol derivatives, castor oil derivatives, siloxanes, or mixtures thereof, or mixtures with coemulsifiers thereof.

29. The method as claimed in claim 23, wherein the agent further comprises an aqueous phase and one or more W/O or O/W emulsifiers or mixtures or mixtures with coemulsifiers.

30. The method as claimed in claim 24, wherein the agent further comprises an aqueous phase and one or more W/O or O/W emulsifiers or mixtures or mixtures with coemulsifiers.

31. The method as claimed in claim 25, wherein the agent further comprises an aqueous phase and one or more W/O or O/W emulsifiers or mixtures or mixtures with coemulsifiers.
